(19) United States
(12) Patent Application Publication
(10) Pub. No.: US 2006/0235211 A1
(43) Pub. Date: Oct. 19, 2006

(54) Lactones of carboxylic acid polysaccharides and methods for forming conjugates thereof

(76) Inventors: Ned D. Heindel, Easton, PA (US); Wallace D. Longton, Boiling Springs, PA (US); Christine Martey-Ochola, Harrisburg, PA (US); Robert D. Rapp, Laurelldale, PA (US)

Correspondence Address: COMPETITIVE TECHNOLOGIES, INC., 1960 BRONSON ROAD, FAIRFIELD, CT, 06824, US

(21) Appl. No.: 11/408,750

(22) Filed: Apr. 21, 2006

Related U.S. Application Data
(63) Continuation-in-part of application No. 09/493,891, filed on Jan. 28, 2000.
(60) Provisional application No. 60/674,343, filed on Apr. 22, 2005.

(51) Int. Cl. C08B 15/06; C07H 5/06; C07H 17/02

(52) U.S. Cl. 536/17.4; 536/55.3; 536/31

(57) ABSTRACT

Novel lactones of polysaccharide carboxylic acids, and methods for the preparation of a variety of conjugates therefrom, as well as the novel conjugates, are provided. Conjugates include metallo-coordinated cisplatin (and carboplatin), as well as conjugates of ellipticinium, aminoglutethimide, mitoxantrone, mitoguazone, cis 3-hexen-1-ol, and other nucleophilic biologically effective agents. Also provided are conjugates via a coupling agent of biologically effective agents, such as Vitamin E, DTPA, TAXOL and TAXOTERE.

This application is a continuation-in-part of co-pending U.S. patent application Ser. No. 09/493,891, filed Jan. 28, 2000, which is incorporated herein in its entirety, and claims the benefit of co-pending U.S. Provisional Patent Application Ser. No. 60/674,343, filed Apr. 22, 2005, which is incorporated herein in its entirety.

### FIELD OF THE INVENTION

The present invention relates to sustained release drug and other compound conjugates, and in particular to carboxymethylcellulose-drug conjugates, and to related carboxylic acid polysaccharide-bioactive compound conjugates.

### BACKGROUND OF THE INVENTION

The treatment of many diseases often involves the administration of drugs. Such drugs may be biocides, as for example anti-bacterial or antiviral drugs, or they may supply missing metabolic intermediates or end-products, or they may inhibit or stimulate a normal metabolic pathway in the body. Not infrequently, such drugs have a limited or local target, and are toxic or have other untoward side effects when active outside the limited or local target. However, most drugs are delivered systemically, or non-specifically, as for example orally (by pills and liquids), by injection, and by transfusion, and reach approximately the same concentration throughout the body. On the other hand, it is difficult to reach certain targets with systemically delivered drugs; as an example, the blood-brain barrier prevents many circulating drugs from reaching the brain. Moreover, once administered, drugs are typically metabolized by the body to an inactive form, and/or excreted, thus exhibiting a short lifetime in the body and requiring frequent doses to maintain an effective concentration at the targeted site.

Therefore, it would be desirable to develop drug delivery systems that could deliver drugs directly to the desired target, or very nearby. This would result in therapeutically effective concentrations at the desired target, but with lower concentrations elsewhere in the body, thereby decreasing adverse systemic effects of drugs. Targeted or local delivery could also result in decreased amounts of drugs needed, thereby decreasing drug costs. It would also be useful to develop drug delivery systems that slowly release a drug from an inactive storage form, thereby increasing the length of the lifetime of a single dose of an administered drug. It would be most useful to develop a drug delivery system that could both deliver a drug directly to a desired target and slowly release a drug from an inactive storage form.

### SUMMARY OF THE INVENTION

Accordingly, the present invention provides novel carboxy- and carboxymethyl-saccharide lactones and methods for the ring-opening of these lactones to prepare a variety of bioactive conjugates thereof.

Thus, it is an aspect of the present invention to provide a series of lactones of polysaccharide carboxylic acids.

It is another aspect of the present invention to provide methods for preparing a variety of conjugates from the series of lactones of polysaccharide carboxylic acids of the present invention.

It is yet another aspect of the present invention to provide a series of conjugates of the series of lactones of polysaccharide carboxylic acids of the present invention.

It is yet another aspect of the present invention to provide pro-compounds for use in methods to deliver and slowly release bioactive compounds from a storage depot of pro-compound.

Thus, in some aspects, the present invention provides a lactone of polysaccharide carboxylic acid according to Formula I: 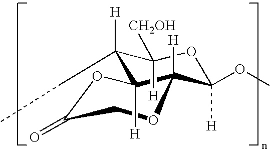
wherein “n” is an integer from 500 to 2000. In further embodiments, “n” is an integer from 1000 to 1500. In yet other further embodiments, the polysaccharide carboxylic acid is selected from the group consisting of carboxy- and carboxymethyl cellulose, carboxy- and carboxymethyl cyclodextrin, carboxy- and carboxymethyl starch, carboxy- and carboxymethyl chitosan, and pectin.

In other aspects, the present invention provides a method for the synthesis of a lactone of polysaccharide carboxylic acids comprising providing a free acid form of the polysaccharide as a finely-powdered, anhydrous carboxylic acid with minimal sodium and potassium carboxylate content, and lactonizing the polysaccharide by thermal dehydration in an anhydrous non-nucleophilic solvent, wherein the polysaccharide is not dextran. In further embodiments, the polysaccharide carboxylic acid is selected from the group consisting of carboxy- and carboxymethyl cellulose, carboxy- and carboxymethyl cyclodextrin, carboxy- and carboxymethyl starch, carboxy- and carboxymethyl chitosan, and pectin. In other further embodiments, the solvent is selected from the group consisting of xylene, toluene, diglyme, and acetonitrile. In some embodiments, the polysaccharide carboxylic acid is carboxymethyl cellulose and the solvent is diglyme. In other embodiments, the polysaccharide carboxylic acid is pectin and the solvent is toluene. In yet other embodiments, the polysaccharide carboxylic acid is carboxymethyl starch and the solvent is diglyme.

In yet other embodiments, the present invention provides a polysaccharide carboxylic acid lactone product made according to the method described above.

In yet other aspects, the present invention provides a method for the synthesis of a polysaccharide carboxylic acid lactone conjugate comprising coupling a polysaccharide carboxylic acid lactone as described above with a bioactive compound, wherein the coupling occurs via ring-opening of the lactone. In further embodiments, the ring opening is accomplished with a metallo bioactive compound. In yet further embodiments, the metallo compound is cisplatin or carboplatin. In other embodiments, the ring opening is accomplished with a nucleophilic compound. In yet further embodiments, the nucleophilic compound is selected from the group consisting of aminoglutethimide, ellipticinium, mitoxantrone, mitoguazone, finasteride, alpha-difluoromethylornithine, and cis 3-hexen-1-ol.

In yet other aspects, the present invention provides a method for the synthesis of a therapeutic compound comprising conjugating a therapeutic agent to a polysaccharide carboxylic acid utilizing a reactive lactone of the polysaccharide carboxylic acid.

In yet other aspects, the present invention provides a method for the synthesis of a conjugated polymer comprising conjugating a bridging nucleophile to a polysaccharide carboxylic acid utilizing a reactive lactone of the polysaccharide carobxylic acid, and attaching an electrophilic bioactive molecule to the bridging nucleophile. In further embodiments, the bridging nucleophile is hydrazine. In other further embodiments, the bioactive molecule is selected from the group consisting of Vitamin E, DPTA, TAXOL, and TAXOTERE.

In other aspects, the invention provides a polysaccharide carboxylic acid lactone conjugate or pro-compound made according to the methods described above. In further embodiments, the polysaccharide carboxylic acid lactone is prepared from a polysaccharide carboxylic acid selected from the group consisting of a carboxy- and carboxymethyl cellulose, carboxy- and carboxymethyl cyclodextrin, carboxy- and carboxymethyl starch, carboxy- and carboxymethyl chitosan, and pectin, and the conjugate is selected from the group consisting of cisplatin, carboplatin, aminoglutethimide, ellipticinium, mitoxantrone, mitoguazone, finasteride, alpha-difluoromethylornithine, cis 3-hexen-1-ol, Vitamin E, DPTA, TAXOL, and TAXOTERE.

### BRIEF DESCRIPTION OF THE FIGURES

FIG. 1 shows the controlled release of Cisplatin from CMC/CMD Cisplatin complexes made and tested according to the present invention and as described in Example 17.

FIG. 2 shows In vivo tumor growth inhibition by ellipticine conjugated to SCMC, as demonstrated by treating JC tumor bearing female Balb/c mice with ellipticine or ellipticine conjugated to SCMC as described in Example 18.

### DEFINITIONS

To facilitate an understanding of the present invention, a number of terms and phrases are defined below. As used herein:

The term “conjugate” means joined together, coupled, or acting or operating as if joined. Conjugates refer to products, and include complexes, such as metal coordinated and metal chelated, as well as compounds ionically or covalently attached. A conjugate may be referred to by one or both of the original compounds joined together to form the conjugate, as for example, a polysaccharide carboxylic acid lactone conjugate.

The term “degree of substitution” or “d.s.” means the ratio of attached molecules per each repeating monomer unit, usually glucose or galactose, in each of the saccharide carriers employed in the present invention.

The terms “conjugation activators” or “chemical promoters” include carbodiimides, mixed anhydrides, homo and hetero-bifunctional couplers and related agents effecting small molecule to macromolecule attachment, as for example is described in Bioconjugate Techniques (by Greg T. Hermanson (1996), Academic Press).

The term “polysaccharide carboxylic acid lactone” refers to a lactone of a polysaccharide carboxylic acid, preferably as synthesized as described in this application. Any polysaccharide carboxylic acid except dextran is included; preferably, the polysaccharides are carboxy- and carboxymethyl cellulose, carboxy- and carboxymethyl cyclodextrin, carboxy- and carboxymethyl starch, carboxy- and carboxymethyl chitosan, or pectin

The terms “biologically efficacious” or “biologically active” or “bioactive” or “active” or the like mean having a biological response or effect or activity within the targeted end-user. When used to describe an agent or compound, such as “bioactive agent” or “bioactive compound,” the phrase means that the agent or compound has a biological response or effect or activity within the targeted end-user.

The term “therapeutic” includes treatment or prevention of any medical condition, for example, those conditions including, but not limited to, malignant and benign conditions, BPH, and endometriosis. A “therapeutic agent” is a compound with a therapeutic effect.

The term “pro-compound” means a conjugate prepared from a polysaccharide lactone and a potentially biologically active molecule, and from which a biologically active molecule can be released. Thus, the active moiety, be it pharmaceutical, flavorant, fragrance or other end-use product, is in a generally inactive form while part of the conjugated material, and becomes ‘active’ when released from the conjugate.

The term “pro-perfume” means a chemical wherein the biologically-active ingredient is a molecule possessing useful fragrance properties ‘tethered’ or conjugated to a polysaccharide from which it demonstrates prolonged release either with or without enzymatic action.

The term “pro-flavor” means the polysaccharide ‘tethered’ or conjugated to a polysaccharide carrier of which it possesses useful flavoring properties when released, with or without enzymatic action, from the carrier.

The term “storage depot” or “storage reserve” refers to a collection of pro-compounds comprising a polysaccharide-bioactive molecule conjugate, from which the bioactive molecule is capable of release in active form, which release is achieved either enzymatically or non-enzymatically over a period of time.

### DESCRIPTION OF THE INVENTION

The present invention relates to sustained release drug and other compound conjugates, and in particular to carboxymethylcellulose-drug conjugates, and to related polysaccharide-bioactive compound conjugates. It is contemplated that, in some aspects of the present invention, polysaccharide-bioactive compound conjugates of the present invention can act as a storage reserve or depot of the bioactive compound which is slowly released from the conjugated form when present in the body of an organism. It is further contemplated that the conjugates of the present invention can be situated locally to provide directed release of the conjugated bioactive compound into a target area. Thus, in other aspects, it is contemplated that some conjugates of the present invention can be delivered by a targeted storage depot, from which the bioactive compounds can be released in a controlled fashion.

The present invention was developed from efforts to prevent and/or treat internal adhesions, such as peritoneal or intra-abdominal adhesions.

The formation of peritoneal or intra-abdominal adhesions is a frequent and often perilous derivative of general abdominal surgery, hernia repair, laparotomy, peritoneal injury, or radiation therapy (Weibel, M A and Majno, G (1973) Am. J. Surg., 126:345-347; Mathes, S J and Alexander, L (1996) Surg. Oncol. Clin. North America 5(4):809-824; and Holmdahl, L et al. (1997) Eur. J. Surg. Suppl. 577: 56-62). Weibel's autopsy report of 752 patients who had undergone abdominal surgery revealed an adhesion rate of 67%. Even surgical division of an adhesion to relieve a previously induced intestinal obstruction may result in a recurrence of the adhesion in as many as 32% of the cases (Brightbill, N L et al. (1997) Arch. Surg., 112: 505). For 1992, the National Center for Health Statistics reported 344,000 operations within the USA alone to repair peritoneal adhesions (Graves, E J (Apr. 8, 1994) National Center for Health Statistics, U.S. Dept. of H.H.S., 249:7). Fibrotic tissue damage resulting from radiation therapy is dose dependent and may not be clinically evident for months or years after treatment (Mathes, S J and Alexander, L (1996) Surg. Oncol. Clin. North America 5(4): 809-824).

These internal adhesions can become pathologic as a result of the anatomical distortions that result. These distortions cause subsequent morbidities such as intestinal obstructions, infertility, chronic pelvic pain, volvulus, or even hemorrhage. Many techniques have been employed to reduce the incidence of adhesion formation induced by radiation or surgery. Such techniques include adjusting the surgical approach, or administration of antioxidants, hyperbaric oxygen, fibrinolytic drugs, phospholipids, or barrier polymers (see Baeum, H et al. (1997) Free Radical Res. 27 (2):127-142; Holmdahl, L et al. (1997) Eur. J. Surg. Suppl. 577: 56-62; and Mathes, S J and Alexander, L (1996) Surg. Oncol. Clin. North America 5(4): 809-824).

Polar, water-soluble, and often anionic barrier polymers seem to be the most successful of these techniques. Such polymers are used in one of two ways. In one, the polymers are used as thin absorbable films mechanically placed on site, where they inhibit vessel growth and prevent adhesion. In another, the polymers are injected as viscous aqueous solutions intraperitoneally (i.p.) such as during laproscopic surgery. Some examples of useful water-soluble anionic polymers—all of which have been shown capable of reducing adhesions—include sodium hyaluronic acid, sodium carboxymethyl cellulose, chondroitin sulfate, heparin, papain, and sodium polyacrylate (Alponat, A et al. (1997) Am. Surgery 63(9): 818-819; J. W. Burns, J W et al. (1996) Fertil. Steril. 66(5): 814-821; Cvetkovic, N et al. (1997) 52(7): 536-537; Harris, E S et al. (1995) Surgery 117(6): 663-669; Oelsner, G et al. (1987) J. Reprod. Med. 32(11): 812-814; Ortega-Moreno, J (1993) Arch. Gynecol. Obstet. 253(1): 27-32; M. Parra, O et al. (1991) Arch. Gastroenterol 28(2): 63-68; and Seeger, J M et al. (1997) J. Surg. Res.68(1): 63-68). Ordinary dextran has been employed, but appears to display the most minimal adhesion protective effect (Harris, E S, et al. (1995) Surgery 117(6):663-669).

Since treatment for malignancy is the most common medical reason for surgery or radiation therapy in the abdominal area, the combination of an anti-adhesion pharmacology with an anti-tumor effect in a polymeric therapeutic adjuvant would represent a profound clinical advantage.

In their use as barrier polymers, carbohydrates have demonstrated low mutagenecity and low allergenicity, and are thus fairly well tolerated in the body. When looking for a platform or carrier for sustained or slow drug release, carbohydrates were thus considered a likely candidate. For example, cyclodextrins have been used to store and release drugs; this use is based upon the ability of cyclodextrins to absorb drugs in their interstices, from which the drugs are able to slowly leach out. However, it is difficult to control the absorption and subsequent release of drugs in this system. Other studies have indicated a dose-related impairment of renal function in some animals (see, for example, Donaubauer, H H (1998) in Regul. Toxicol. Pharmacol. 27 (2): 189-98, and Jameson, C W (1992) Toxic Rep. Ser. 14, 1-C2).

A possible solution is to link drugs to the carbohydrate polymers. However, current techniques used to link compounds to carbohydrates require the use of reagents and reactants, many of which possess or result in undesirable characteristics, especially in the resulting carbohydrate-drug linked product. As a result, such products are generally unsuitable for use in a patient's body.

As one example, many reactants are nitrogen based, and are therefore allergenic. Use of typical conjugation activators or chemical promoters may leave unproductive, N-acyl rearranged residues from the promoter itself on the polymer backbone. In this context, the terms “conjugation activators” or “chemical promoters” includes carbodiimides, mixed anhydrides, homo and hetero-bifunctional couplers and related agents effecting small molecule to macromolecule attachment, as for example is described in Bioconjugate Techniques (by Greg T. Hermanson, Academic Press (1996)). With covalent attachment of molecules to carboxyl groups by the traditional carbodiimide-promoted coupling, a substantial number of unproductive, N-acyl rearranged residues attached to the polymer backbone often result (Heindel, N D et al. (1994) Bioconjugate Chem 5: 98-100). These residues have the potential to contribute to the immunogenicity and the toxicity of the saccharide polymeric carrier.

As another example, many reactions for linking molecules to carbohydrates require the use of excess reactants, resulting in residuals left in the resulting product. Since most couplings are traditionally performed in aqueous media, the inherent instability to water of the coupling promoters must be compensated by use of an excess quantity of promoter (Gilles, M A et al. (1990) Anal. Biochem. 184:244-248). In addition, a chemical residue of the reagents intended to promote coupling often remains on the macromolecule backbone (Wong, S S (1991) Chemistry of Protein Conjugation and Cross-Linking (CRC Press) pp. 122-123) and is difficult to remove. Thus, even if both the polysaccharide carrier and the agent being released from it are acceptable as being safe, as reflected either by inclusion on the GRAS list or on the FDA's list of approved pharmaceuticals, the altered carriers containing the residues of the non-productive coupling agents may have an adverse, or at least an unknown, pharmacology.

A solution to the problem of chemical residues of promoters such as carbodiimide is to not utilize them in the first place. A highly reactive internal lactone prepared from carboxymethyl dextran and employed in coupling without any promoting agents being present has been reported (Heindel, N D et al. (1994) Bioconjugate Chem 5: 98-100). However, dextran glucose polymers are unsuitable for use as implants as slow release drug storage depots or reservoirs, as they are very rapidly metabolized in vivo. Cellulosic polysaccharides have a much longer half-life in vivo. Moreover, as noted above, dextran also appears to display the most minimal adhesion protective effect (Harris, E S, et al. (1995) Surgery 117(6):663-669).

It was not believed that other polysaccharides other than dextran would be amenable to lactonization for several reasons. Dextran is a very atypical carbohydrate, as it is highly water soluble and flexible and slippery. In contrast, most other polysaccharides are insoluble, or only slightly soluble, in water, and are much more rigid molecules. Thus, it was unexpected to discover that other polysaccharides are also amenable to lactonization; however, it is necessary to use different reaction conditions to produce the lactone products. It was then further discovered that these other polysaccharide lactones can be conjugated to bioactive or other active compounds, without the use of any coupling catalysts or promoters.

Accordingly, the present invention provides novel polysaccharide lactones, and methods for the ring-opening of these lactones to prepare a variety of biologically-efficacious conjugates thereof.

In one aspect, the present invention provides series of lactones of polysaccharide carboxylic acids. In further aspects, the present invention provides a series of lactones of carboxy- and carboxymethyl-saccharides.

In another aspect, the present invention provides methods for preparing a variety of conjugates from the series of lactones of polysaccharide carboxylic acids.

In yet another aspect, the present invention provides a series of conjugates of the series of lactones of polysaccharide carboxylic acids of the present invention. In further aspects, the conjugates comprise biologically active compounds conjugated to the polysaccharide carboxylic acids of the present invention.

It is yet another aspect of the present invention to provide pro-compounds for use in methods to deliver and slowly release bioactive compounds from a storage depot of pro-compounds.

In some embodiments, the present invention provides methods whereby a reactive lactone prepared from a carboxyl-containing polysaccharide such as carboxymethyl cellulose can be linked directly to a biologically active molecule (as, for example, an anti-tumor drug) containing an amino or hydroxyl function, or to any nucleophilic species, without use of the typical conjugation activators or chemical promoters. These methods provide significant advantages, in that the linkage to the carbohydrate is clean, and without significant residuals.

In other embodiments, the present invention provides methods whereby a reactive lactone of polysaccharide can be linked indirectly to a bioactive molecule, where the bioactive molecule is an electrophile; in these methods, a bridging group, such as a bi-functional nucleophile, may be inserted between the polysaccharide lactone and the condensing electrophilic bioactive molecule.

The methods of lactone opening of the present invention provide several advantages over current methods that utilize typical conjugation activators or chemical promoters. These advantages include but are not limited to avoiding the necessity of excess quantities of coupling promoters, and avoiding the presence of chemical residues of these promoters on the macromolecular backbone. Moreover, the method of the present invention for attaching molecules to polysaccharide lactones never utilizes all the available lactones, and therefore upon hydrolysis, and pH adjustment, pendant acid carboxyls or carboxylate salts result. As noted above, carboxylate salts provide an advantage of inhibiting adhesion formation during wound repair.

In different aspects of the present invention, lactones of many different polysaccharides can be prepared and coupled to biologically active molecules for therapeutic, flavoring or fragrance applications without any intervening chemical promoter. For example, as noted above, anionic polysaccharide carboxylates are useful in anti-adhesion therapy. Thus, in one aspect of the present invention, whereby pharmaceuticals are conjugated to anionic polysaccharide carboxylates according to the present invention, the conjugates can be used as both anti-adhesion compounds and cancer chemotherapeutics with utility in healing abdominal malignancies that have been surgically excised or treated by radiation in situ. In this aspect, the present invention provides the advantage of the combination of an anti-adhesion pharmacology with an anti-tumor effect in a polymeric therapeutic adjuvant. This provides a particular benefit in treating microfocal malignancies left behind after surgery to remove tumors.

In a more detailed description of the present invention, processes for the preparation of polysaccharide lactones, processes for the preparation of chemotherapeutic and biologically active conjugates prepared from these lactones, and applications of the conjugates are described in more detail below. Applications of these conjugates include, but are not limited to, drug-bearing lactones to chemoprophylaxes of adhesions arising in oncologic therapies.

I. Novel Carboxy- and Carboxymethyl-Saccharide Lactones

A. Starting Polysaccharides

Suitable polysaccharides for use in the present invention include any mono- or polysaccharide with both carboxyl (—COOH) and hydroxyl (—OH) groups, where the molecule is sufficiently flexible for the carboxyl and the hydroxyl to bond internally into a lactone. Exemplary carboxy- and carboxymethyl-saccharide lactones include but are not limited to those derived from carboxylic acid containing cellulose, starch, cyclodextrin, citosan, and pectin. Polysaccharides that yield internal lactones according to the present invention include carboxymethyl cellulose, carboxymethyl cyclodextrin, carboxymethyl starch, carboxymethyl chitosan, pectin, and carboxy starch. The carboxymethylated saccharides are widely reported and synthesized by traditional condensation of the parent carbohydrate with chloroacetic acid in aqueous base. The degree of substitution (d.s.) of available —COOH per repeating monomeric carbohydrate unit in the saccharide acid preferably does not exceed 1.2, although much lower d.s. (0.25 to 0.80) performs well. Optimum lactonization is achieved when the carboxylic group is able to statistically grip a proximal hydroxyl, or in other words, there is at least one hydroxyl group within “gripping distance” of a carboxyl group to form a lactone.

While carbohydrate derivatives are well known to those skilled in this art, it is best to note alternative names, and in some cases, commercial suppliers. Carboxymethylcellulose sodium salt, also known as SCMS or CMCS, is available from Hercules Inc. (Wilmington, Del.); pectin potassium salt is available from Sigma (St. Louis, Mo.) (fruit pectin conventionally sold to the public for use in home canning is also satisfactory if purified as noted herein; O,N-Carboxymethyl, 0-carboxymethyl, and N-carboxymethylchitosan are available from CarboMer (Westborough. Mass.). and N-carboxymethylchitosan is available from V-Labs Inc. (Covington, La.) (these chitosan products can also be found in the literature as glucosamine polymer, carboxylmethylated on 0-, N-, or mixed 0,N-specified); carboxymethyl starch is also known as starch glycolate or as starch carboxymethyl ether and is available as its sodium salt from Penwest Pharmaceuticals (Patterson, N.Y.) or National Starch Inc. (Bridgewater, N.J.); carboxymethyl alpha- and beta-cyclodextrins are available as the free acids from CarboMer; carboxy-starch is a research-grade product produced by partial oxidation of the C-6 primary hydroxyl on starch by National Starch Inc.

B. Preparation of Lactones

The general route of lactone synthesis involves the conversion of polysaccharide to its acid form, and of the polysaccharide acid to its lactone. The process of lactonization can be followed by the infrared C=0 shift from acid (ca 1720 cm⁻¹) to lactone.

1. Purification of Starting Polysaccharides

Preferably, saccharide acids are purified, finely-powdered, anhydrous carboxylic acids with minimal sodium or potassium carboxylate content. By “minimal” it is meant that about 5 percent or less of the carboxyls are present in their sodium or potassium salt form, and that about 95 percent or greater are present as free acid forms. The free acid form of the carbohydrate generates a lactone under the methods of the present invention.

To obtain effective lactonization, starting materials (whether indicated as free acids or as salts by method of synthesis or by label description on commercial materials) are dissolved in distilled, deionized water and passed over a mixed bed resin ion exchange column. Exemplary but non-limiting columns include a Sigma Mixed Bed Resin TMD-8 column and a Baker Ionac NM-60 Mixed Bed Resin. The eluant is then dialyzed, and the dialyzate dried. Exemplary dialysis includes but is not limited to use of a dialysis bag, as for example Sigma 12,000 molecular weight exclusion. Exemplary but non-limiting dialyzate drying methods include evaporation in vacuo and/or lyophilization. The presence of carboxylate anion and of free carboxylate acid forms of the polysaccharides can be evaluated, as for example by FT-IR spectra (carboxylate anion, C=0 ca 1610 cm⁻¹; free carboxylic acid forms, C=0 in the range of 1720-1645 cm⁻¹ depending on extent of internal hydrogen bonding). The resulting samples are reduced to a fine powder, as by for example, grinding, ball-milling, or “wiggle-bug” reduction, and the samples may be stored, preferably to under vacuum over a drying agent, until lactonization is performed.

2. Lactonization

The general lactonization synthetic method involves heating finely pulverized acid in an anhydrous non-nucleophilic solvent. Evaporation and filtration yield the lactone. This general method is applicable to all carboxylic acid saccharides.

Lactonization is carried out by thermal dehydration in an anhydrous non-nucleophilic solvent. The acid can be lactonized either as a suspension of the insoluble acid or as a solution or partial solution. Well-stirred media such as refluxing mixed xylenes (bp 138-144° C.), toluene (bp 109-110° C.), diglyme (bp 162° C.), and acetonitrile (bp 82° C.) perform satisfactorily. Degree of substitution (d.s.) of available —COOH per repeating monomeric carbohydrate unit in the saccharide acid preferably does not exceed 1.2, although much lower d.s. (0.25 to 0.80) performs well. Carboxymethyl moieties lactonize more extensively than the less flexible, more constrained, directly attached carboxylic acid moieties such as found in pectin acid and 6-carboxystarch.

Although the reaction temperature is not critical, heating is preferably done at about the natural reflux temperature of the solvent. Thus, in some embodiments, heating is accomplished at temperatures from about 80° C. to about 165° C., with the reaction generally completed in shorter times at higher temperatures. In preferred embodiments, the reaction is agitated during heating by mechanical or magnetic stirring of the suspended solid to prevent a darkening of the mass when caking against a heated surface occurs. The ratio of saccharide carboxylic acid to solvent may be varied, from about 1 gm of solid to from about 15 ml to about 100 ml of solvent. Reaction progress can be monitored by withdrawing a portion of the reaction mixture for analysis; exemplary sample sizes are about 15 mg of solid, though other amounts are possible, and exemplary analysis is spectral analysis, as described herein. The reaction is considered completed when the carboxylic acid peak has vanished and the lactone peak has reached maximum intensity. Typically, with 1 gm of starting material in 25 ml of refluxing, well agitated xylene, the reaction is complete in about 60 min when heated at about 138-140° C. In other embodiments, about 1 gm of finely pulverized acid is heated at about 160-162° C. in about 50 ml of vigorously stirred anhydrous diglyme for up to about 24 hours.

A chemical structure ‘flow chart’ of these two general reactions, purification and lactonization, for obtaining lactones of the present invention is:

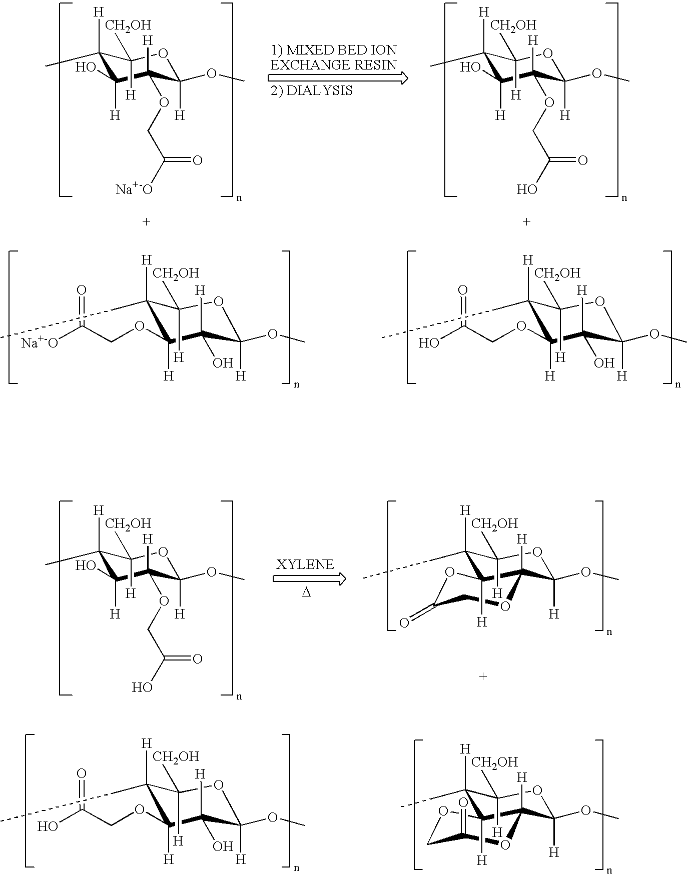

In the above structures, the exact number of repeats as indicated by ‘n’ is not critical to the present invention, and thus ‘n’ may vary over a wide range, depending upon the characteristics of the final product sought. However, ‘n’ may normally be an integer from 500 to 2000, preferably between 1000 and 1500. The range is well within the skill of those in the art to determine given the description of the present invention herein and the knowledge of what specific properties of the final conjugate are desired for a specific use.

As recognized by those skilled in the art, lactones can be recognized by altered physical properties compared to their starting acids. Carboxymethyl cellulose lactone, for example, deposits from the lactonization solvent as a film which can be pulverized to a yellowish-white solid that is virtually insoluble in water; carboxymethyl dextran lactone can be collected as a water-insoluble white solid.

In their infrared spectra, all lactones display a long wavelength C=0 between 1740 and 1760 cm−1. On a high-resolution FT infrared spectrophotometer, the band envelope of the lactone C=0 stretch is often seen to consist of several closely spaced absorptions, presumably reflecting the presence of several different specific lactone structures. Thus, the process of lactonization can be followed by the infrared C=0 shift from acid (ca 1720 cm⁻¹) to lactone. Lactones open and dissolve in aqueous base.

II. Methods of ring-opening saccharide lactones and preparation of conjugates thereof.

In further aspects of the present invention, the reactive lactone of polysaccharides prepared as described above can be coupled to a bioactive compound, wherein the coupling occurs via ring-opening of the lactone. In some embodiments, coupling occurs in a mixture of the polysaccharide lactone and the bioactive molecules; these include molecules capable of forming metallo complexes and molecules capable of covalent attachment to the carboxyl function. In other embodiments, the polysaccharide lactone is first reacted with a bridging molecule, and the bioactive molecule than linked to the bridging molecule. Starting materials for these reactions are described in further detail below.

Linkage to polysaccharide lactones of the present invention as described above includes but is not limited to metallo complexes whose attachment can be coordinated to hydroxyl groups, and to compounds capable of covalent attachment to the carboxyl function by a hydrolyzable bond. The latter set of compounds preferably bear an amino, hydroxyl, or mercapto group which can link to the lactones and thereby open them up.

Exemplary but non-limiting examples of metallo complexes include those of pharmaceutical merit, including aminoplatimun (II) complexes used in cancer chemotherapy; this family of platinum complexes includes carboplatin and cisplatin. Other complexes include zinc pyrithione, an antifungal agent, and magnesium acetylsalicylate, an analgesic/antipyretic.

Preferred compounds conjugated to polysaccharide lactones of the present invention include bioactive compounds. “Biologically-efficacious” or “biologically active” or “bioactive” or “active” or the like means having a biological response or effect or activity within the targeted end-user.

Exemplary biologically efficacious conjugates include but are not limited to: therapeutics, including but not limited to metallo-coordinated cisplatin (and carboplatin) conjugates and covalently linked conjugates of ellipticinium, aminoglutethimide. mitoxantrone, finasteride, alpha-difluoromethylornithine (DFMO), mitoguazone (also known as MGBG or methylglyoxalbisguanylhydrazone) and other nucleophilic chemotherapeutics, and electrophilic therapeutic or other bioactive compounds which can be linked to the carbonylhydrazide prepared from the lactone, such as vitamin E, TAXOTERE, and TAXOL, and estrone; imaging diagnostic compounds, such as saccharide bound chelating agents capable of binding radioactive metal ions for nuclear imaging or paramagnetic metal ions for magnetic resonance imaging, such as DTPA (N,N,N′,N″,N″-diethylenetriaminepentaacetic acid); fragrances for application in, for example, laundry or washing products; flavorings for application in, for example, foods and chewing gums; and property modifiers, including but not limited to thickeners, humectants, dispersants, in, for example, foods, paints, and other products. In sum, carboxy-functionalized and carboxymethyl-functionalized polysaccharide conjugates and compounds produced from the lactones according to the present invention have utility in a wide range of products.

Direct condensation of any molecule with the lactones according to the present invention requires that the incoming reactant be a nucleophile; in preferred embodiments, the nucleophile is a small molecule. Exemplary but non-limiting nucleophiles include amines and alcohols which readily open the lactones. The only limitation on structure is that the nucleophilicity and steric accessibility of the —NH or —OH functions in these biologically active molecules be sufficient to ring-open the saccharide lactones. Thus, in some embodiments, any amino or hydroxy containing biologically-active compound can be conveniently linked to the lactone of carboxymethyl cellulose (CMCL), the lactone of carboxymethyl dextran (CMDL), the lactone of carboxymethyl starch (CMSL), the lactone or pectin, or any of the other lactones according to the present invention.

For those cases in which it is desirous to attach an electrophile, a bridging bi-functional nucleophile may be inserted between the polysaccharide lactone and the condensing electrophile to be attached; exemplary but non-limiting examples of a linking agent that fulfills this bridging function include hydrazine, ethylenediamine, and 1,3-diaminopropane.

The use of a bridging group is one example of a “spacer group” between a pharmaceutical and a carrier in the controlled release and in targeted drug delivery. In addition to the carboxyhydraxzide spacer (described in more detail below), other spacer groups between drug and carrier include dibasic acids (Deutsch, H M et al (1989) J. Med. Cham. 32: 788-792; Safavy, A et al. (2003) Bioconjugate Chem. 14: 302-310).

In some embodiments, biologically active substances may be pharmaceuticals, including but not limited to adriamycin, daunomycin, gemcitabine, bleomycin, 6-mercaptopurine, 5-FU, mephalan, ellipticine (and ellipticiniums such as ellipticinium bromide), mitoguazone, aminoglutethimide, squalamine, mitoxanthone, alpha-difluoromethylornithine, podophyllotoxin, and methotrexate.

In addition, such biologically active substances include flavoring alcohols, including but not limited to leaf alcohol (cis-3-hexen-1-ol), menthol, acetoin (3-hydroxy-2-butanone), thymol, vanillin, and methyl salicylate. Attached as a pro-flavor to gum, candy, food, or any nutritional or non-nutritional product placed in the oral cavity in the form of a conjugate of the saccharides according to the present invention, these chemicals sustain their biological activity (i.e., providing flavor) over a prolonged time, whether being released from the saccharide with or without enzymatic action.

Furthermore, the biologically active substances also include fragrance compounds, including but not limited to leaf alcohol (cis-3-hexen-1-ol), phenylethyl alcohol, 3-methyl-5-phenyl-1-pentanol, 2-methyl-5-phenyl-1-hexanol, 1-hexanol, 1-decanol, 1-dodecanol, 3,7-dimethyM-octanol, isononanol (i.e., 3,5,5-trimethylhexanol and isomers), 2,2-dimethyl-3-phenyl-1-propanol, nopol, anisic alcohol, benzyl alcohol, 2-cyclohexylethyl alcohol, 2,4-dimethylcyclohexylmethanol, beta-methylphenylethyl alcohol, hydroxycitronellol, isocyclogeraniol, 3-hydroxymethyl-2-nonanone, 4-isopropylbenzyl alcohol, 3-phenylpropanol, and others.

III. Utility

A. Procompounds

After lactonization according to the method of the present invention, and subsequent linkage of a bioactive molecule to the polysaccharide lactone, each of the unique polymeric conjugates of the present invention is capable of releasing the “active ingredient.” Furthermore, by controlling the effective degree of substitution (i.e., drug or other ‘active ingredient’ moieties per repeating glucose unit) at the time of synthesis, the period of in vivo release of such conjugated compounds can be adjusted. The carboxy and carboxymethyl polysaccharide conjugates made in accordance with the method of the present invention, or in other words those compounds to which have been attached an appropriate “active ingredient” or “biologically active” molecule, have uses in therapeutics, including but not limited to body implants as well as areas outside of outside of therapeutics, including but not limited to formulations for laundry products, chewing gum, food processing, and paint improvers.

In some embodiments of biologically active agents having therapeutic activity, two major classes of chemotherapeutics with established efficacy against malignancies of the abdominal regions were selected for exemplary linkage to carboxymethyl cellulose as pro-drugs (as conventionally understood). These chemotherapeutics are (1) those metallo complexes whose attachment is coordinated (cisplatin, carboplatin), and (2) those capable of covalent attachment to the carboxyl function by a hydrolyzable bond (for example, the anticancer drugs ellipticine [acyl hydrazide], mitoxantrone [amide], aminoglutethimide [amide], and mitoguazone [amide]) or via a linking agent (for example, vitamin E [ester] and TAXOL ester and TAXOTERE [ester]), where the hydrolysable linkage is indicated in brackets after each compound. Chemical coupling of the biologically active agent (pharmaceutical) to the lactone provides a sustained release formulation (a pro-drug) that is released both with and without enzymatic action.

In other embodiments of the present invention, saccharide lactone chemistry according to the present invention provides a useful method for the preparation of pro-perfumes and pro-flavors. In these embodiments, a “pro-perfume” is a chemical wherein the biologically-active ingredient is a molecule possessing useful fragrance properties “tethered” or conjugated to a polysaccharide from which it demonstrates prolonged release either with or without enzymatic action. A “pro-flavor” is the polysaccharide “tethered” or conjugated to a polysaccharide carrier of which it possesses useful flavoring properties when released, with or without enzymatic action, from the carrier.

B. Product Formulation

For the formulation of suitable products from the conjugates according to the present invention, as described above and below, and for all the mentioned carboxy- and carboxymethyl-saccharide conjugates, nonreacted lactone is hydrolyzed with base and dialyzed to neutrality, after which the conjugate is retained as an aqueous solution, lyophilized powder, gum, or film. Since all conjugates retained uncoupled carboxylic acid moieties, they may be adjusted to their sodium salts by pH adjustment following well known protocols. This variability gives flexibility in the form for use of the conjugates according to the present invention.

Forms for use comprise, but are not limited to, injectable aqueous solutions as pro-drugs for therapy, mechanically implanted strips as pro-pharmaceutical depots, ribbons or films employed in fulfillment of the clinical objectives of adhesion inhibition or malignant growth suppression in intraperitoneal cavities, solid or solution additives as pro-flavors to food, and solid or solution additives as pro-perfumes to laundry products. Of course, by referring to these conjugates as “pro-” compounds, it is meant that the active moiety, be it pharmaceutical, flavorant, fragrance or other end-use product, is in a generally inactive form while part of the conjugated material, and becomes ‘active’ when released from the conjugate. For example, in some embodiments, conjugates of vitamin E may be used to promote local or system wound healing. In other embodiments, conjugates of N,N,N′,N″,N″-diethylenetriaminepentaacetic acid (DTPA) may be used as either their gadolinium chelates for magnetic resonance imaging (MRI) or as their radiometal chelates for nuclear medicine imaging.

### EXAMPLES

These and other aspects of the present invention can be deduced by those skilled in the art to which it pertains by reference to the preceding description and the following Examples. The following Examples are thus provided for purposes of clarity in order to more fully describe and demonstrate the methods by which the lactones and conjugates according to the present invention are prepared. However, these Examples are not meant to be limiting in any manner, and modifications and adaptations may be made to provide other routes or end products, all of which are to be considered to be within the scope of the present invention.

In the Examples, sodium carboxymethyl cellulose, carboxymethyl cellulose (acid form), and the in situ hydrolyzed carboxymethyl cellulose (lactone form) were used to demonstrate linkage according to the present invention for the exemplified metallo-coordinated cisplatin.

In addition to the chemotherapeutic examples according to the present invention, conjugates of fragrance and flavor components were used to demonstrate linkage of biologically active compounds. Thus, the employment of cis-3-hexen-1-ol (or “leaf alcohol”) ‘tethered’ or conjugated to a polysaccharide carrier is described as exemplary of the attachment of a further flavor or fragrance ingredient according to the present invention.

### Abbreviations

In the Examples, the following abbreviations are used:
bp (boiling point); ° C. (degree Centigrade); d.s. (degree of saturation); hr (hour); i.p. (intraperitoneally); mp (melting point);
BPH (*); CMC (carboxymethyl cellulose); CMD (carboxymethyl dextran); CMCS or SCMS (Carboxymethylcellulose sodium salt); CMCL (lactone of carboxymethyl cellulose;
CMDL (lactone of carboxymethyl dextran), CMSL (lactone of carboxymethyl starch); DFMO (alpha-difluoromethylornithine); DTPA (N,N,N′,N″,N″-diethylenetriaminepentaacetic acid);
EDCI (3-dimethylaminopropyl)-3-ethylcarbodiimide hydrochloride); EEDQ (1-ethoxycarbonyl-2-ethoxy-1,2-dihydroquinoline); 5-FU (*); IR (infrared); FT (*); MGBG (mitoguazone or methylglyoxalbisguanylhydrazone); MRI (magnetic resonance imaging);
NMR (nuclear magnetic resonance); UV (ultra violet); Vis (visible)

### EXAMPLE 1

### Preparation of the Lactones

a) Purification of Starting Materials

It is preferred that all saccharide acids be purified, finely-powdered, anhydrous carboxylic acids with minimal sodium or potassium carboxylate content. Only the free acid form of the carbohydrate generates a lactone under the conditions according to the present invention. To obtain effective lactonization, all starting materials (whether indicated as free acids or as salts by method of synthesis or by label description on commercial materials) were dissolved in distilled, deionized water and passed over a mixed bed resin ion exchange column. While other appropriate columns would be satisfactory, the Sigma Mixed Bed Resin TMD-8 column was selected for this purpose. The eluant was charged to a dialysis bag (Sigma 12,000 molecular weight exclusion), and dialyzed against distilled water for three to five days with replacement of the external water every 24 hours. The contents of the dialysis bag were evaporated in vacuo and lyophilized for 24 hours. FT-IR (FT=? IR=infrared) spectra showed no trace of carboxylate anion (C=0 ca 1610 cm⁻¹) but only the free carboxylic acid forms (C=0 in the range of 1720-1645 cm⁻¹ depending on extent of internal hydrogen bonding) of the polysaccharides. Grinding, ball-milling, or “wiggle-bug” reduction to a fine powder was performed and the samples were held under vacuum over a drying agent until lactonization was performed.

b) Lactonization

Lactonization was carried out by thermal dehydration in an anhydrous non-nucleophilic solvent. The acid can be lactonized either as a suspension of the insoluble acid or as a solution or partial solution. Well-stirred media such as refluxing mixed xylenes (bp 138-144° C.), toluene (bp 109-110° C.), diglyme (bp 162° C.), and acetonitrile (bp 82° C.) perform satisfactorily. Degree of substitution (d.s.) of available —COOH per repeating monomeric carbohydrate unit in the saccharide acid should not exceed 1.2, although much lower d.s. (0.25 to 0.80) performs well. Carboxymethyl moieties lactonize more extensively than the less flexible, more constrained, directly attached carboxylic acid moieties such as are found in pectin acid and 6-carboxystarch.

A chemical structure ‘flow chart’ of these two general reactions for obtaining the lactonization according to the present invention is:

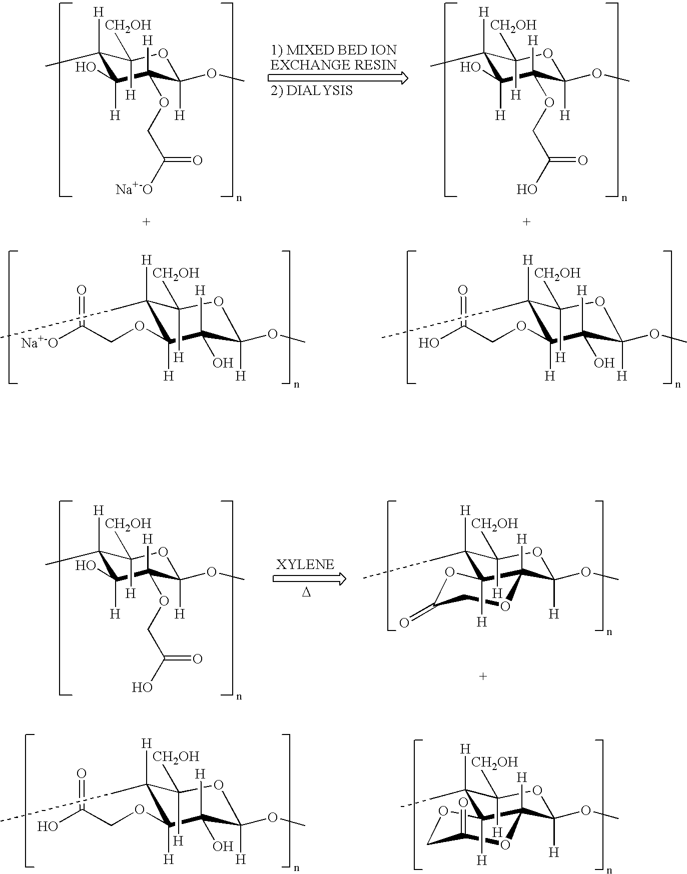

In the above structures, the exact number of repeats is not critical to the present invention, and thus ‘n’ may vary over a wide range, depending upon the characteristics of the final product sought. However, ‘n’ may normally be an integer from 500 to 2000, preferably between 1000 and 1500. The range is well within the skill of those in the art to determine given the description of the present invention herein and the knowledge of what specific properties of the final conjugate are desired for a specific use.

As recognized by those skilled in the art, lactones can be recognized by altered physical properties compared to their starting acids. Carboxymethyl cellulose lactone, for example, deposits from the lactonization solvent as a film that can be pulverized to a yellowish-white solid that is virtually insoluble in water; carboxymethyl dextran lactone was collected as a water-insoluble white solid.

In their infrared spectra all lactones display a long wavelength C=0 between 1740 and 1760 cm⁻¹. On a high resolution FT-IR spectrophotometer, the band envelope of the lactone C=0 stretch is often seen to consist of several closely spaced absorptions presumably reflecting that several different specific lactone structures are present. Lactones open and dissolve in aqueous base. The general lactonization synthetic method requires heating about 1 gram of the finely pulverized acid in 50 ml of vigorously stirred anhydrous diglyme for 24 hours. Evaporation and filtration yield the lactone. This general method is applicable to all carboxylic acid saccharides according to the present invention.

In the following Examples, carboxymethyl cellulose was completely lactonized, carboxymethyl dextran was about 90% lactonized, and pectin acid was about 50-70% lactonized.

### EXAMPLE 2

### Preparation of Carboxymethyl Cellulose Lactone (CMCL)

The hitherto unknown lactone of carboxymethyl cellulose (CMCL) provides a reliable coupling promoter for a wide variety of amino- and hydroxyl-containing biologically important molecules. The route to the synthesis of this lactone is illustrated below, wherein the sodium carboxymethyl cellulose is converted to the acid and the acid to the lactone.

Carboxymethyl cellulose (free acid) was purified as previously described. The white flaky solid (1.0 g) was pulverized to dust in a wiggle-bug, suspended in 60 ml of anhydrous diglyme (or xylene), and heated at 150° C. for 24 hrs. The solvent was evaporated to 25 ml, chilled, and the lactone filtered off as a water-insoluble off-white solid. This solid was filtered and washed quickly twice with 10 ml each of cold water. The resulting product was then dried in vacuo by lyophilization.

If all the solvent used in the lactonization is evaporated to dryness in a rotary vacuum evaporator, then the lactone can be obtained as a film clinging to the walls of the vessel. A characteristic C=0 stretch was seen at 1750 cm⁻¹ using FT-IR and is indicative of this lactone. The process of lactonization can be followed by the infrared C=0 shift from acid (ca 1720 cm⁻¹) to lactone.

### EXAMPLE 3

### Preparation of Pectin Lactone

A suspension 2.0 g of purified, dried, finely pulverized pectin acid was prepared in 70 ml of anhydrous toluene and heated with stirring at reflux for 24 hr, following the general procedure described above. Evaporation of the solvent yielded a water-insoluble, gummy, semi-solid whose IR spectrum revealed lactone (1748 cm⁻¹) and nonreacted acid (1680 cm⁻¹) in an intensity ratio of 70/30 lactone/acid. While the pectin acid could not be driven to a higher lactone content, this lactone could be ring-opened with nucleophilic molecules (i.e., primary and secondary amines and alcohols); in preferred embodiments, the nucleophile is a small molecule. Alternatively, as described below, it was possible to optimize the loading of the small molecule onto pectin by an in situ generation and ring-opening of the lactone.

### EXAMPLE 4

### Preparation of Carboxymethyl Starch Lactone (CMSL)

Sodium carboxymethyl starch (1.0 g) was converted to the free acid, purified, dried, and pulverized as described above. It was lactonized by refluxing in 60 ml of anhydrous diglyme, isolated and purified as described above. FT IR spectra on the sodium salt displayed the carboxylate —COO— at 1620 cm⁻¹ and on the purified acid displayed the C=0 at 1717 cm⁻¹. Lactonization after 24 hr reflux was greater than 90% complete and the new lactone C=0 was evident at 1742 cm⁻¹.

### EXAMPLES 5-7

### Preparation of Carboxymethyl Cellulose-Cisplatin Complex

In complexometric binding of platinum (and indeed other metal-containing complexes) to carboxylic acid ligands, ion concentrations may be critical. For example, it has been reported that cisplatin associates best with carboxylic residues if the sodium ions have been removed and all the carboxylic moieties are in the acid form (Schechter, B et al. (1986) Cancer Biochem. Biophys. 8: 277-287). Therefore, additional time and effort is needed to convert the clinical grade of sodium carboxymethyl cellulose (SCMC) to the non-sodium containing free acid.

In the following Examples 5-7, the preparation of the cisplatin complex with COOH (the acid form of the polymer), with COONa (sodio salt form of the polymer), or with in situ hydrolyzed lactone is described. The sodium carboxymethyl cellulose (SCMC) has a molecular weight of about 250,000 and a degree of substitution (d.s.) of about 0.8 to about 0.9.

Although the procedures described below were carried out with various chemical forms of carboxymethyl cellulose, the techniques are applicable to other carboxy- and carboxymethylated polysaccharides and their lactones as described herein.

### EXAMPLE 5

### Preparation of Carboxymethyl Cellulose-Cisplatin Complex From the Acid-Form of the Polymer

2.5 grams of sodium carboxymethyl cellulose (SCMC) were dissolved in 100 ml of distilled/deionized water by heating and agitation at about 80-90° C. for approximately 10 minutes. The solution remained homogeneous on cooling to room temperature after which it was passed through an ion exchange resin column (Sigma: Mixed Bed Resin TMD-8), dialyzed (Sigma: membrane 12.000 mw exclusion) for three days with three exchanges of water, evaporated in vacuo to about one-fifth the volume (about 20 ml). The resulting solution was then divided into two equal 10 ml portions, one of which was lyophilized, dried to constant weight, and weighed. This technique was used to determine the number of grams (or moles) of the cellulosic polymer in the remaining aqueous aliquot.

In a separate sequence, 15-30 mg of cisplatin was dissolved in 1 to 2 ml of distilled/deionized water by briefly heating and agitating at about 80-90° C. A pale yellow homogeneous solution resulted. The solutions of cisplatin and carboxymethyl cellulose were then mixed in a volume ratio to ensure that a mole ratio of 10/1 cisplatin/—OCH₂COOH was employed. Moles of carboxyl moieties on the cellulose were determined from the known degree of substitution. The solution was sealed and stirred at room temperature for 24 hours, during which time a clear, nearly colorless solution resulted. Dialysis for removal of unbound cisplatin was carried out against distilled/deionized water for five days with two water exchanges. After dialysis was completed, the solution within the bag was diluted with distilled/deionized water to 25.0 ml. Since there is no loss of the carboxymethyl cellulose in the purification process, and since the final fluid volume is known, one can calculate the moles of cisplatin to moles of the polymer by experimentally determining the amount of bound cisplatin. Exhaustive dialysis in this fashion removed sodium ions and produced the final polymer as carboxymethyl cellulose (CMC)-cis-platinum adduct.

An established analytical method for cisplatin bounded to polymeric systems (Schechter, B et al. (1986) Cancer Biochem. Biophys. 8: 277-287) was employed in which o-phenylenediamine was utilized as a quantitative chromogen for a complex which was read at 703 nm against a calibration standard curve. A typical complex prepared in this fashion has about 40 to about 50 millimoles of cisplatin per mole of CMC. The degree of substitution can be varied over a wide range (for example, of from about 20 to about 400 millimoles) of cisplatin (or carboplatin) per mole of CMC by control of the initial reacting ratio of the drug to carboxymethyl functions. Furthermore, use of a polymer is not limited to one whose (d.s.) is as low as about 0.8 to about 0.9, and carboxyl loads of from about 1.4 to about 1.8 are serviceable. With higher (d.s.) of carboxyls, higher (d.s.) of cisplatin (or carboplatin) linkage are achieved.

### EXAMPLE 6

### Preparation of Carboxymethyl Cellulose-Cisplatin Complex From the Sodium Salt-Form of the Polymer

2.5 grams of SCMC was dissolved in 100 ml of distilled/deionized water by heating and agitation at about 80-90° C. for approximately 10 minutes. The solution was then cooled to room temperature and evaporated in vacuo to about 20 ml. It was then mixed with a cisplatin solution as described above (with the understanding that the ratio of platinum to carboxyl and of carboxyls per repeating glucose moiety can be varied widely) and treated in the same manner as the free acid. No significant differences in load of cisplatin/cellulose unit were observed whether commencing with the free acid or the carboxylate salt.

### EXAMPLE 7

### Preparation of Carboxymethyl Cellulose-Cisplatin Complex From the Lactone of Carboxymethyl Cellulose (CMCL)

It is also possible to utilize the internal lactone (synthesized as described above) by heating, with agitation, at between about 80-90° C., a suspension of 2.5 g of the CMCL mixed with an appropriate amount of cisplatin, as described above. The lactone, which is incompletely soluble in water, dissolves and reacts with the cisplatin, apparently as its carboxylic residues are hydrolytically generated. No sodium ions and no base need be present in this drug-loading process. These are important features and aspects of utilizing a lactone in forming such complexes. When commencing with the lactone, the same load of cisplatin/cellulose was achieved as when using the free acid or the carboxylate salt.

### EXAMPLES 8-12

### Conjugation of Nucleophilic Biologically Active Substances to the Lactones

Direct condensation of any molecule with the lactones according to the present invention requires that the incoming reactant be a nucleophile; preferred nucleophiles are small molecules. Amines and alcohols readily open the lactones. Condensing the nucleophiles according to the present invention is described below.

The following examples describe the versatility of the conjugates according to the present invention, and provide procedures for linking four pharmaceuticals (pro-drugs) and one perfume flavorant (pro-fragrance and pro-flavor).

### EXAMPLE 8

### Preparation of CMC-Aminoglutethimide Conjugates

CMC-lactone (CMCL) according to the present invention was prepared as previously described, and was opened by the amino function on the aminoglutethimide moiety. 360 mg of aminoglutethimide were dissolved in 40 ml of anhydrous acetonitrile and 336 mg of CMCL were added to form a suspension. The suspension was refluxed for 18 hrs under argon, after which the reaction was cooled and the excess solvent removed under reduced pressure. Excess drug was removed by dissolving the cream colored flakes in 3 ml 0.1 N NaOH, adding 7 ml of distilled water, and dialyzing the solution against distilled water for 5 days with 2 changes of water. The small quantity of aqueous base opens the residual lactone moieties that had not undergone reaction with the drug. After dialysis, the retentive (sample in bag) was concentrated under reduced pressure, and final drying was accomplished by lyophilization.

The proton NMR and FT-IR show clear evidence that both drug and polymer are present. As was noted with cisplatin, the ratio of available drug per available lactone permits variation in the degree of substitution that is determined, in this case, by combustion analysis for nitrogen.

1H NOR (DO) 0.72 (t), 1.76 (m), 2.15 (m), 2.25 (m), 2.9-4.3 (Br), 6.85 (DD), 6.91 (DD). FT-IR (KBr pellet): 1730 cm⁻¹ (acid), 1650 cm⁻¹ (Br, amide). mp>300° C. d.s.=0.10 by method above; if reactant ratios are varied, d.s. values of 0.01 to 0.5 are obtained when the original saccharide had 0.85 carboxymethyls/glucose.

### EXAMPLE 9

### Preparation of CMC-Ellipticinium Conjugate

120 mg of 2-(4-hydrazino-4-oxobutyl) ellipticinium bromide (yellow powder) was dissolved in 25 ml anhydrous acetonitrile, and 235 mg CMCL added leading to formation of a suspension. The reaction was stirred and refluxed under argon for 15 hr, and the excess solvent removed by evaporation in vacuum. The deep yellow flakes were only partially soluble in water due to nonreacted lactone. A few drops of 0.1 N NaOH resulted in complete solubility by opening the remaining lactone. The excess ellipticine was dialyzed against water for 5 days with 2 changes of water. The yellow retentive was concentrated under reduced pressure and dried via lyophilization.

Quantitation for (d.s.) was performed against an ultra-violet calibration curve prepared with the ellipticinium standard at the indicated maximum. Proton NOR and FT-IR showed the presence of drug and polymer. The 0.13 load of ellipticinium on the conjugate made full interpretation of its peaks impossible with the larger contribution of the polysaccharide. Aromatic resonances in the conjugate from 6.6 to 8.9 matched the aromatic protons in the original drug. C—H resonances from the carbohydrate obscured the remaining portion of the spectrum. The original ellipticinium possesses an unsymmetrical carboxy hydrazide C=0 at 1652 cm⁻¹ and the CMCL possesses its C=0 at 1750 cm⁻¹, whereas the new conjugate displays C=0 bands at 1728, 1694 and 1652 and none at 1750 cm⁻¹.

1H NOR (DO) 1.52-1.54, 1.79, 1.96, 3.47-4.60(Br),4.11, 6.66. 7.24, 7.64, 7.79, 7.95, 8.95 ppm. FT-IR (KBr disk): 1694, 1651 cm⁻¹. UV (DO): 430 (Br weak), 365 (Br weak), 305 (strong), 245 (weak) nm. mp>300° C. d.s.=0.13 by the method above; by variation of reaction ratios a range of (d.s.) from 0.01 to 0.4 can be achieved.

### EXAMPLE 10

### Preparation of CMC-Mitoxantrone Conjugates

80 mg of mitoxantrone hydrochloride salt (blue powder) were dissolved in 30 ml of anhydrous acetonitrile upon addition of 2 equivalents of triethylamine. 145 mg of CMCL were added, and the reaction was carried out at reflux for 22 hr under argon. The acetonitrile was evaporated off, and the excess mitoxantrone was dialyzed against a 0.001 N solution for 5 days with 2 changes of solution. This is done so as to ensure that the non-attached mitoxantrone transverses the dialysis bag because it is not very soluble in water. The retentive obtained after dialysis against the NaOH solution was further dialyzed for 3 days in distilled water with 2 changes of water to remove excess. The CMC-mitoxantrone conjugate (blue flakes) was dried via lyophilization.

Quantification was carried out against a calibration curve prepared with authentic mitoxantrone.

1H NOR (DO); 1.09 (m), 2.85-3.93 (Br), 6.75 (d). FT-IR (KBr disk): 1720, 1645, 1600, and 1590 cm⁻¹. UV (water); several weak bands, maximum at 604 nm, mp>300° C., d.s.=0.1 (range obtainable by adjusting reaction ratios 0.01 to 0.25).

### EXAMPLE 11

### Preparation of the CMC-Mitoguazone Conjugate

A 100 ml round bottom flask was charged with a suspension of 75 mg (408 mmol) of mitoguazone, 50 mg of CMC-lactone (or CMCL), and 10 ml of anhydrous acetonitrile. The reaction medium was refluxed and stirred for 24 hours, filtered, and the solid residue washed on the filter with a minimum of cold water. The solid residue was briefly dried in vacuum to ensure removal of the organic solvent (residual acetonitrile can dissolve holes in the dialysis bag into which the conjugate was dissolved in 80 ml of distilled water). Dialysis was conducted for five days with three changes of the external water. Lyophilization produced an off-white conjugate of no-defined melting point (decomposition over a temperature range in excess of 100° C.). Mitoguazone itself acts like a pH indicator, changing from a clear, nearly colorless solution to a bright yellow solution as the pH is raised from the acid range to 11. The purified carboxymethyl cellulose conjugate of mitoguazone evidenced this same pH-related color change also at pH 11. Even though this color transition can be spectroscopically quantified by weighed standards of the free drug and used to determine degree of substitution, the most sensitive determination is by combustion analysis to quantify nitrogen content of the dried conjugates. The parent CMCL, of course, has no nitrogen content.

The procedure described above gives a product with 7.43 nitrogen, which reflects a d.s. of 0.15. When carried out with a greater excess of mitoguazone, or for a longer reflux time, or in higher boiling solvents (diglyme, bp 162° C.; dioxane, bp 101° C.), higher d.s. values of 0.15 to 0.30 can be obtained.

### EXAMPLE 12

### Preparation of the Pectin Lactone Conjugates of Leaf Alcohol

a) From Pre-Prepared Pectin Lactone

A charge of 540 mg of pectin lactone and 20 ml of leaf alcohol (cis 3-hexen-1-ol) was placed in a 50 ml round bottom flask fitted with a magnetic stirrer. Although insoluble at ambient temperature, most of the pectin lactone dissolved, i.e., reacted, when heated to reflux. Reflux and stirring was continued for 24 hr, whereafter the excess leaf alcohol was removed by heating in vacuo. The resulting gum was dissolved in a minimum quantity of cold methanol and filtered to remove insoluble material. Upon evaporation in vacuo, the polymer was examined by IR spectroscopy, revealing about a 50% esterification. No lactone remained.

b) By In situ Lactonization and Esterification on Pectin

Into a 25 ml flask fitted with a magnetic stirrer and condenser was placed 8.0 ml of leaf alcohol (cis 3-hexen-1-ol). The alcohol was warmed to 50° C. and treated to the portionwise addition of 200 mg of purified, dried, powdered pectin acid. The temperature was raised to 60° C., and 4.0 ml of anhydrous toluene were added. The mixture was then heated to distill volatiles from the medium. 4 ml of additional toluene were added to replace that which distilled and the distillation continued. After 6 hrs of heating, the mixture was cooled to room temperature and a solid carbohydrate precipitated (IR showed few ester and many carboxylic acid carbonyls in the solid). The solid precipitate was washed on the filter with 10 ml of hexane, and the combined organic fractions distilled (30° C. at 0.6 Torr) to remove both hexane and leaf alcohol. The light yellow oil was shown by FT-IR to possess 50 ester/acid (1718 cm⁻¹ ester C=0 and 1670 cm⁻¹ pectin acid C=0).

### EXAMPLES 13-16

### Conjugation of Electrophilic Biologically Active Substances to the Lactones

For those cases in which it is desirous to attach an electrophile, a bridging bi-functional nucleophile may be inserted between the polysaccharide lactone and the condensing electrophile to be attached; exemplary but non-limiting linking agents that fulfills this bridging function include hydrazine, ethylene diamine and 1,3-propanediamine.

All lactones react rapidly and in high efficiency with hydrazine (all lactones present are opened). By way of illustration of this condensation process, the preparation of a hydrazide from CMCL and hydrazine is described in Example 13. Examples 14 and 15 vitamin E (as alpha-tocopherol sulfo-N-hydroxysuccinimido succinate) and N,N,N′,N″,N″-diethylenetriaminepentaacetic acid (as DTPA bis-anhydride) coupled to this hydrazide.

It has also been discovered that polysaccharide lactone-to-hydrazide pathway (Example 13), applied to conjugation of vitamin E and DTPA (Examples 14 and 15), can also be utilized for the conjugation of TAXOL and TAXOTERE (Example 16). Taxane derivatives are well known to possess potent in vivo antitumor efficacy in a wide variety of malignancies (for review, see Taxol: Science and Applications (M. Suffness, ed (1995) CRC Press, Boca Raton, 426 pp.) Controlled release of TAXOL analogs either from a depot macromolecule or a soluble form of a conjugate is contemplated to find wide clinical utility in extending the biological half-life in vivo.

### EXAMPLE 13

### Preparation of Hydrazide From CMCL and Hydrazine

The reaction of CMCL with hydrazine was initiated by mixing 10 mg of CMCL, 5.0 ml of hydrazine hydrate, and 3.0 ml of water. The lactone quickly dissolved. The solution was allowed to stand for 3 hrs, diluted with 20 of water and dialyzed for 5 days with 3 changes of water. The solid hydrazide obtained on evaporation and lyophilization displayed FT IR bands at 1058 and at 1594 cm⁻¹ characteristic of —CO—NH—NH₂. Combustion analysis for nitrogen gave 6.09 N, which translates to a d.s. of 0.48.

### EXAMPLE 14

### Preparation of Vitamin E Conjugates

A solution was prepared from 33.0 mg, 0.045 mmoles, of the vitamin E derivative, sodium alpha-tocopherol-sulfo-N-hydroxysuccinimide (Molecular Biosciences) in 7.0 ml of water, which was filtered to remove traces of insoluble material. The fluid volume was increased to 15 ml by the addition of distilled water, and 10 mg of carboxymethylcellulose hydrazide, pre-dissolved in 7.0 ml of water, were added. The resulting solution was stirred and heated at 50° C. for 24 hrs, and then at 25° C. for a second 24 hr period, during which a white precipitate of the vitamin E conjugate formed.

Quantitative UV spectroscopy showed that the d.s. of vitamin E on the carboxymethylcellulose hydrazide was 0.1.

### EXAMPLE 15

### Preparation of the Diethylenetriaminopentaacetic Acid Conjugate

20 mg, 0.056 mmoles, of the bis-anhydride of diethylenetriaminopentaacetic acid (DTPA), 10 ml of anhydrous pyridine, and 10 mg of carboxymethylcellulose hydrazide were charged to a 25 ml round bottom flask and heated to 115° C. for 24 hrs. The cellulose hydrazide did not dissolve until 10 ml of distilled water were added. Evaporation in vacuum and dialysis of the crude solid (5 days, 3 water changes to remove the non-conjugated DTPA) gave the conjugate after lyophilization. The d.s. was 0.05.

### EXAMPLE 16

### Preparation of TAXOL and TAXOTERE Conjugates

It is contemplated that the hemisuccinates of TAXOL analogs, and in particular TAXOL (Paclitaxel) and TAXOTERE (Docetaxel) hemisuccinates, capped with oxysuccinimidate moieties, link directly to the polysaccharide hydrazides described in Example 13. Thus, the linkage of TAXOL (Paclitaxel) and TAXOTERE ((Docetaxel) is through a hemi-succinate bi-functional bridge using an ester bond from the dug to the bridge. This differs from all of the preceding exemplified linkages, where the linkage is via an amino group and hence the linkages are from amide or hydrazide functions off the pro-compound (except for Vitamin E in Example 14).

While there exists a family of TAXOL analogues, TAXOTERE (also known as Docetaxel) and TAXOL (also known as Paclitaxel) are the most commonly referenced antitumor agents.

TAXOL (PACLITAXEL) (structure 1 below)

TAXOTERE (DOCETAXEL) or (structure 2 below)

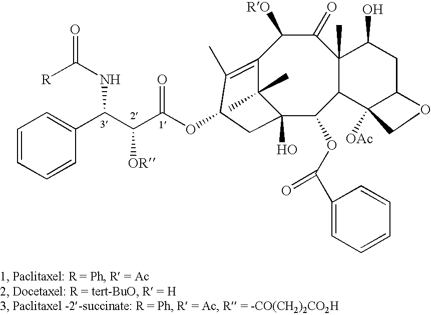

A general method has been described by Deutsch for preparing the hemisuccinates (structure 3) from the 2′-hydroxy on the side chain of Paclitaxel (Deutsch, H M, et al. (1989) J. Med. Chem., 32: 788-792); this procedure is equally applicable to Docetaxel. Furthermore, Safavy has described how the carboxylic acid terminus of structure 3 can be converted to an oxysuccinidimate for facile displacement by nucleophiles (Safavy, A, et al. (2003) Bioconjugate Chem. 14: 302-310). Safavy applied this concept to coupling Paclitaxel to Erbitux, a monoclonal antibody, via backbone amino groups (presumably mostly lysines) on the protein framework using a transesterification promoter (1-ethoxycarbonyl-2-ethoxy-1,2-dihydroquinoline, also known as EEDQ) and N-hydroxysuccinimide (Safavy, A, et al. (2003) Bioconjugate Chem. 14: 302-310).

It is contemplated that 3-dimethylaminopropyl-3-ethylcarbodiimide hydrochloride (EDCI) can perform the same function.

It is further contemplated that Paclitaxel and Docetaxel hemisuccinates, capped with oxysuccinimidate moieties, link directly to the polysaccharide hydrazides described above in Example 13.

Thus, the general synthetic scheme consists of preparing a hemisuccinate derivative of TAXOTERE, a hydrazide derivative of carboxymethyl cellulose, and the condensation of these two derivatives.

a) Procedures:

Commencing with sodium carboxymethylcellulose (degree of substitution=0.9), the lactone was prepared as described in Example 1, and the lactone ring opened by two different procedures to generate the carboxyhydrazide. Procedure A (an aqueous reaction) generated a mixture of carboxylic acids and carboxyhydrazides on the carbohydrate backbone, while Procedure B (an anhydrous reaction) generated only carboxy hydrazide functionalities. Procedure C describes the coupling methodology.

Procedure A (Aqueous Reaction to Generate Carboxyhydrazide)

In the first procedure, hydrazine hydrate in water was used exactly as described in Example 13. For the resulting reaction product, combustion analyses for nitrogen indicated a degree of substitution of 0.45 hydrazides per repeating glucose unit, indicating that the polysaccharide bears a nearly equal number of —CO—NH—NH₂ moieties (IR bands 1057 and 1598 cm⁻¹) with —COOH moieties (several IR bands falling between 1718 and 1645 cm⁻¹ but centered on 1690 cm⁻¹, reflecting different hydrogen-bonded species). No lactone C═O bands (1755±10 cm⁻¹) remain.

Procedure B (Anhydrous Reaction to Generate Carboxyhydrazide)

A substantially improved variation of Procedure A employs anhydrous hydrazine—generated by a drying process employing calcium hydride—in acetonitrile solvent, as described in more detail below. For the resulting reaction product, combustion analyses for nitrogen indicate a quantitative conversion of all lactone functions to —CO—NH—NH₂ functions. The indicated carbonyl hydrazide bands as reported above were observed, with no remaining lactone C═O bands, and barely detectable —COOH bands in the infrared spectrum. Thus, this improved procedure maximizes the hydrazide content.

Into an Erlenmeyer flask containing 1.0 g (24 mmol) of CaH₂ and 10 ml of dry CH₃CN was placed 1.0 ml (20.4 mmol) of hydrazine hydrate in a dropwise fashion. The flask was cooled in a water bath during the addition. After the drying reaction has ceased, the suspension was filtered through a Teflon Millipore membrane and the filter cake rinsed with a small amount of dry CH₃CN. The filtrate was transferred to a 25 ml round bottom flask containing a magnetic stir bar and 59.8 mg (0.296 me) of pulverized CMC lactone. The flask was closed with a glass stopper and tightly sealed with Parafilm. The mixture was stirred at room temperature for 6 days.

The insoluble material was filtered off and washed with absolute EtOH. The dried recovered product amounted to 61.9 mg, corresponding to a 97% conversion. An IR spectrum confirmed that the product was the expected hydrazide. A sample was sent for elemental analysis and matched theory for the sesqui-hydrate of the hydrazide at one hydrazide per available lactone C═O.

For: C₈H₁₄O₆N₂1.5 H₂O % N Calc'd=10.72%; % N Found=11.12%

Since the maximum possible drug load was desired, the carboxymethylcellulose hydrazide with the highest (0.9) degree of substitution was employed for the conjugation studies.

Procedure C (the Coupling Methodology)

As a model procedure for the coupling of Paclitaxel to the carboxymethylcellulose hydrazide, the chemistry was first optimized with benzyl alcohol as a surrogate for Paclitaxel.

i. Benzyl Alcohol

Benzyl alcohol was reacted, according to a standard method, with succinic anhydride to prepare the half-ester (“hemisuccinate”) of succinic acid, otherwise known as benzyl hemisuccinate (Borlinghaus, K P et al. (1987) Cancer Research 47: 4071-4075). A mixture of 52.1 mg (0.25 mmol) benzyl hemisuccinate, 54.3 mg (0.25 mmol) N-hydroxysulfosuccinimide sodium salt, 47.9 mg (0.25 mmol) 1-(3-dimethylaminopropyl)-3-ethylcarbodiimide hydrochloride (EDCI) and 0.60 ml of dry DMF was stirred overnight at ambient temperature.

A solution of 58.6 mg (0.25 meq) CMC hydrazide in 5.0 ml of distilled water was prepared, and to this was added the solution of activated benzyl hemisuccinate from above. The reaction was allowed to proceed for 75 minutes during which time a precipitate formed. The reaction mixture was filtered and the filtrate placed in a vacuum desiccator over concentrated sulfuric acid. After 48 hours, the solid residue was triturated three times with ether to yield a hygroscopic white powder amounting to 147 mg. An FT-IR spectrum (KBr) displayed the expected ester peak at 1734 cm⁻¹ and the carbohydrazide peak at 1598 cm⁻¹.

ii. Paclitaxel

The carboxy-activated Paclitaxel is prepared in phosphate buffered saline (25 nM, pH 8.1) using a 1:2:2 ratio of Paclitaxel hemisuccinate (structure 3) to 2-ethoxy-1-ethoxycarbonyl-1,2-dihydroquinoline (EEDQ) to N-hydroxysuccinimide at 25° C. according to the method of Safavy. (Safavy, A, et al. (2003) Bioconjugate Chem. 14: 302-310). Upon filtration, the crude conjugate, carboxy-activated Paclitaxel, is added directly to the carboxymethyl cellulose hydrazide in a mole ratio calculated to be equimolar in conjugate to available —CO—NH—NH₂ moieties in phosphate buffered saline.

After 10 hours of incubation and gentle stirring, the medium is dialyzed to remove salts and small molecules, and then lyophilized to a dried powder. The method is equally applicable to Paclitaxel and Docetaxel, and for both Paclitaxel and Docetaxel conjugates a d.s. of about 0.05 to 0.20 can be obtained.

b) The Analysis (of the Degree of Substitution of Hydrazide Carboxyls)

Nitrogen combustion analyses can determine the degree of substitution of hydrazide carboxyls per repeating glucose unit on the carbohydrate backbone (as reported above). However, it is believed that a better method for determining the drug load onto those hydrazides is generally hydrolysis followed by UV-Vis spectrophotometry. The results of analysis of direct UV-Vis analysis of the drug load are described in various Examples above. The use of UV-Vis spectrophotometry was possible for those situations in which the drug chromophore was distinct from any UV-Vis absorption of the polymer. However, where there is there is substantial spectral overlap in the UV-Vis with the —CO—NH—NH₂ chromophore, as is the case with simple benzenoid chromophores, an alternative analysis, such as nitrogen combustion analyses, is preferred.

c) Results

i. Determination of Amount of Benzyl Alcohol Coupled to CMC Hydrazide

A weighed amount of the dried coupled product, 15.1 mg (0.0356 meq), was stirred overnight in 3 ml of 6N HCl at room temp. The resulting solution was extracted 5 times with 3 ml of methylene chloride, and the extracts combined, placed in a 10 ml volumetric flask, and evaporated under a stream of nitrogen at room temperature. The residue was taken up in 95% EtOH and diluted to the mark. The absorbance was determined at 206 nm and the concentration obtained from a standard curve prepared with benzyl alcohol.

The degree of substitution was 24%, which represents a minimum value.

ii. Determination of Amount of Paclitaxel or Docetaxel Coupled to CMC Hydrazide

When this analysis technique is applied to the Paclitaxel or Docetaxol conjugates, a degree of substitution (d.s.) of 0.05 to 0.20 is observed.

d) Utility

The polysaccharide-Paclitaxel or polysaccharide-Docetaxol conjugates can be applied directly onto or into a tumor, and/or as a supplement to systemic therapy. Before the invention of the technology described in the present application, there were no methods for applying such chemotherapeutics directly onto or into a targeted site. The utility and efficacy of the present technology is confirmed in Examples 17-18.

### EXAMPLE 17

### Bioavailability of Bound Drug

The cisplatin bound to the cellulose carrier polymer according to the method of the present invention as described above was established as being bio-available both by enzymatic action (freshly drawn rat serum) and by spontaneous hydrolysis (pH 7.4 phosphate buffer).

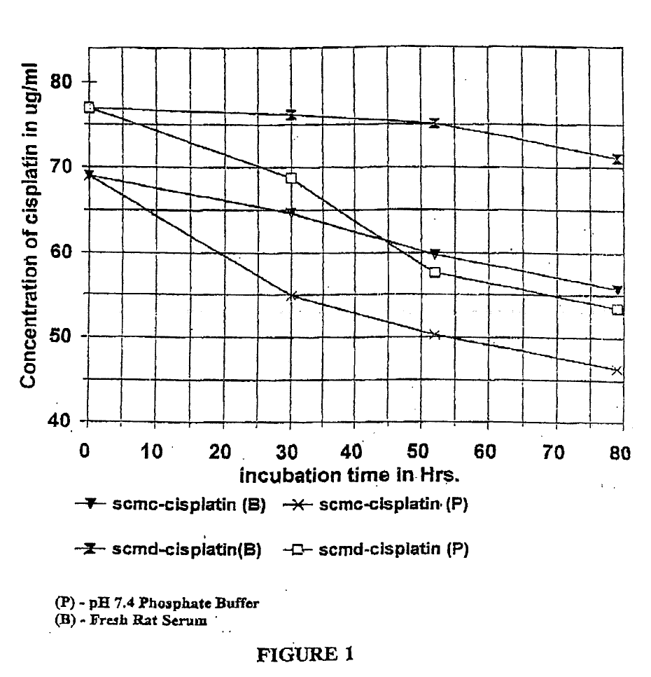

In these procedures, 1.0 ml of rat serum or 1.0 ml of phosphate buffer was mixed with 1 ml of cisplatin-CMC complex (of known concentration and known d.s.) and incubated at 37° C. for 80 hours. The cisplatin continued to be released from the polymer for the entire time span (and beyond) for both the carboxymethyl cellulose (CMC) conjugates (CMC) and the carboxymethyl dextrin (CMD) conjugates in both the rat serum and the phosphate buffer, as shown in FIG. 1.

At the times indicated in FIG. 1, samples were spun against a molecular weight cut-off barrier of 10,000 in a Centricon ultracentrifugation tube, with a platinum analysis performed on the filtrate according to accepted testing protocols. The half-life for drug release in contact was serum was determined to be approximately 8.3 days, and the half-life for drug release in phosphate buffer was determined to be approximately 5.8 days. The drug was released from the dextran carrier in serum with a half-life greater than 16 days.

With specific regard to the bioavailability of drugs from the conjugates according to the present invention, in the experimental fashion described above for measuring the controlled release of cisplatin from the polymer, each of the chemotherapeutic polymer conjugates was evaluated. In freshly prepared mouse serum, the enzyme-mediated release rates of ellipticinium, aminoglutethimide and mitoxantrone were quantified at their ultraviolet maxima according to recognized protocols, and fell in the range of 4-8 day half lives.

### EXAMPLE 18

### Tumor Growth Inhibition

To confirm the therapeutic efficacy of ellipticine conjugated to CMC, mice inoculated with JC cells were subsequently treated with the cytotoxic drug, ellipticine, with or without conjugation to CMC.

As noted above, almost all chemotherapeutics, and especially antineoplastic medications, are administered through the circulatory system, either by intravenous injection or by oral administration. However, treatment of many malignancies would be greatly improved with better control of local drug concentration; non-limiting exemplary malignancies include glioblastoma multiformi, sarcoma, recurrent prostate cancer, and ovarian cancer. One theoretical approach is to attempt control the local drug concentration with interstitial (direct) chemotherapeutic implantation (in other words, by intratumoral injection or application), or other application at the surface or very near vicinity of the tumor. A major difficulty with this approach is finding a slow release mechanism for the drug, while also maintaining a stable localized position of the drug.

The present invention solves this problem by coupling the chemotherapeutic to a polysaccharide carboxylic acid via a lactone intermediate of the polysaccharide carboxylic acid. It is anticipated that use of such a conjugated antineoplastic drug with intratumoral administration will decrease the circulatory concentration of the drug without sacrificing the therapeutic dose necessary in the tumor to achieve tumor growth inhibition and cell death. It is further contemplated that interstitial administration of chemotherapeutic drugs coupled to polysaccharide polymers will provide appropriate treatments to assist cancer patients attain longer lives with a better quality.

Example 9 describes the preparation of CMC-ellipticinium conjugate. Ellipticine, a topoisomerase II inhibitor, has been shown to exhibit strong antineoplastic activity in vitro, but has displayed numerous systemic toxicities in vivo. This Example 18 demonstrates that administering the ellipticine intratumorally is effective, and is anticipated to limit the toxicities that have been associated with ellipyivine in the past.

Materials and Methods

Cell culture and cell lines. Cell line CRL-2116 (ATCC designation: JC) was obtained from the American Type Culture Collection (Manassas, Va.) and cultured as recommended. Cells were maintained in RPMI 1640 (Life Technologies, Inc., Rockville, Md.) with L-glutamine containing 10% FBS and 50 μg/ml gentamicin at 37° C. and 5% CO₂ in 100 mm×20 polystyrene tissue culture dishes.

CMC-ellipticinium conjugate was prepared as described in Example 9.

In vivo growth and treatment. Animal care and procedures were in accordance with guidelines and regulations of the Institutional Animal Care and Use Committee of The Penn State College of Medicine. Balb/c female mice (Charles River Laboratories, Inc.), 6-8 weeks old, were housed (5 per cage) under 12 h light/dark cycles with food and water provided ad libitum. The animals were injected in the subcutaneous space of the right hind quarter with 10⁶ JC cells suspended in PBS. After palpable tumor growth, approximately 3-4 weeks after injection, tumor volume was determined (day 1) using calipers measuring the length (L) and width (W) of the tumor. Tumor volume was calculated using the equation: (L×W²)/2.

The animals were randomized into four treatment groups (n=3-4 animals per group): 1) control (CMC only); 2) ellipticine only; 3) 15% conjugate; and 4). 7.5% conjugate. Treatment was initiated on day 1, and consisted of either an intratumoral administration (30 μL/injection) of CMC alone [what concentration], 9-methoxyellipticinium hydrazide at a dose of approximately 92 mg/kg, a 15% solution of 9-methoxyellipticinium hydrazide conjugated to CMC, or a 7.5% solution of conjugated drug. Tumor injection consisted of three separate injections, approximately 1.25 cm in length (25 gauge needle). Tumor volumes were monitored until day 14 when the animals were euthanized. One-way analysis of variance (ANOVA) test was performed using GraphPad InStat version 3.01 for Windows 95 (GraphPad Software, San Diego, Calif.).

Results

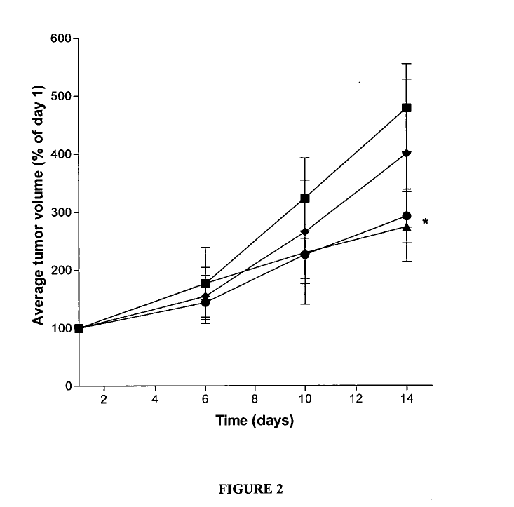

JC tumor bearing female Balb/c mice treated with ellipticine or ellipticine conjugated to CMC were performed as described above in “Materials and Methods.” The results are shown in FIG. 2. Tumor volumes for control (▪), ellipticine alone (▴), 15% solution of ellipticine conjugated to SCMC (●) or 7.5% solution of ellipticine conjugated to SCMC (♦) treated animals are shown as the mean±SD (n=3-4) of the percentage of the day 1 volume. *, P<0.05.

As shown in FIG. 2, the control group experienced an increase in tumor volume of 400% by day 14. Animals treated with 7.5% solution of the conjugated ellipticine demonstrated an increase in tumor size of 300%. In contrast, animals treated with ellipticine alone or the 15% solution of conjugated drug experienced tumor growths of only 150% by day 14 (P<0.05). Thus, administration of ellipticine, either alone or conjugated to CMC, decreased tumor growth rate.

These results, which show in vivo tumor growth inhibition by ellipticine conjugated to CMC, demonstrate the therapeutic efficacy of an antineoplastic drug conjugated to a polysaccharide carboxylic acid via a lactone intermediate of the polysaccharide.

While the preferred embodiment of the invention have been described and illustrated by way of non-limiting examples, it is to be understood that this invention is capable of variation and modification, and it is not intended to be limited to the precise terms set forth, but rather intended to include such changes and modifications which may be made for adapting the present invention to various uses and conditions. Accordingly, such changes and modifications are properly intended to be within the full range of equivalents, and therefore within the purview of the following claims. The terms and expressions which have been employed in the foregoing specification are used as terms of description and not of limitation, and thus there is no intention, in the use of such terms and expressions, of excluding equivalents of the features shown and described, or portions thereof; the scope of the invention being defined and limited only by the claims which follow.

What is claimed is:

1. A method for synthesizing a pro-compound comprising coupling a reactive polysaccharide carboxylic acid lactone with a bioactive compound, wherein the coupling occurs via ring-opening of the lactone.

2. The method of claim 1, wherein the polysaccharide carboxylic acid lactone is synthesized by a method comprising:
(i) providing the free acid form of the polysaccharide as a finely-powdered, anhydrous carboxylic acid with minimal sodium and potassium carboxylate content;
(ii) lactonizing the polysaccharide by thermal dehydration in an anhydrous non-nucleophilic solvent; and
(iii) collecting the resulting lactone product,
wherein the polysaccharide carboxylic acid is selected from the group consisting of carboxy- and carboxymethyl cellulose, carboxy- and carboxymethyl cyclodextrin, carboxy- and carboxymethyl starch, carboxy- and carboxymethyl chitosan, and pectin.

3. The method of claim 2, wherein the solvent is selected from the group consisting of xylene, toluene, diglyme, and acetonitrile.

4. The method of claim 1, wherein the ring opening is accomplished with a metallo bioactive compound.

5. The method of claim 4, wherein the metallo bioactive compound is selected from the group consisting of cisplatin and carboplatin.

6. The method of claim 1, wherein ring opening is accomplished with a nucleophilic bioactive compound.

7. The method of claim 6, wherein the nucleophilic bioactive compound is selected from the group consisting of aminoglutethimide, ellipticinium, mitoxantrone, mitoguazone, finasteride, alpha-difluoromethylornithine, and cis 3-hexen-1-ol.

8. A method for synthesizing a therapeutic compound comprising:
conjugating a therapeutic agent to a polysaccharide carboxylic acid utilizing a reactive lactone of the polysaccharide.

9. A method for synthesizing a conjugated polymer comprising:
(i) conjugating a bridging nucleophile to a polysaccharide carboxylic acid utilizing a reactive lactone of the polysaccharide;
(ii) and attaching an electrophilic bioactive molecule to the bridging nucleophile.

10. The method of claim 9, wherein the bridging nucleophile is hydrazine.

11. The method of claim 10, wherein the bioactive molecule is selected from the group consisting of Vitamin E, N,N,N′,N″,N″-diethylenetriaminepentaacetic acid (DPTA), paclitaxel, and docetaxel.

12. A method for synthesizing a therapeutic compound comprising:
(i) conjugating a bridging nucleophile to a polysaccharide carboxylic acid utilizing a reactive lactone of the polysaccharide; and
(ii) attaching an electrophilic therapeutic compound to the bridging nucleophile

13. A pro-compound made according to the method of any of claims 1-12.

14. A polysaccharide carboxylic acid lactone conjugate, wherein the polysaccharide carboxylic acid lactone is obtained from the group consisting of carboxy- and carboxymethyl cellulose, carboxy- and carboxymethyl cyclodextrin, carboxy- and carboxymethyl starch, carboxy- and carboxymethyl chitosan, and pectin, and wherein the bioactive compound is obtained from the group consisting of cisplatin, carboplatin, aminoglutethimide, ellipticinium, mitoxantrone, mitoguazone, finasteride, alpha-difluoromethylornithine, cis 3-hexen-1-ol, Vitamin E, N,N,N′,N″,N″-diethylenetriaminepentaacetic acid (DPTA), paclitaxel, and docetaxel.

15. The conjugate of claim 14, wherein the polysaccharide carboxylic acid lactone is obtained from carboxymethyl cellulose, and the bioactive compound is ellipticinium.

16. The conjugate of claim 14, wherein the polysaccharide carboxylic acid lactone is obtained from carboxymethyl cellulose, and the bioactive compound is selected from the group consisting of paclitaxel and docetaxel.
